(19) United States
(12) Patent Application Publication
(10) Pub. No.: US 2022/0005553 A1
(43) Pub. Date: Jan. 6, 2022

(54) SUPPORTING METHOD, PRODUCING METHOD AND COMPUTER READABLE STORAGE MEDIUM

(71) Applicant: ASAHI KASEI KABUSHIKI KAISHA, Tokyo (JP)

(72) Inventors: Daisuke HOTTA, Tokyo (JP); Yuki SUGA, Tokyo (JP); Tomotaka HASHIMOTO, Tokyo (JP); Noboru KUBOTA, Tokyo (JP)

(73) Assignee: ASAHI KASEI KABUSHIKI KAISHA, Tokyo (JP)

(21) Appl. No.: 17/363,429

(22) Filed: Jun. 30, 2021

(30) Foreign Application Priority Data
Jun. 30, 2020 (JP) 2020-113514

(51) Int. Cl. G16C 20/30; C13B 20/16

(52) U.S. Cl. CPC G16C 20/30; C13B 20/165

(57) ABSTRACT

Provided is a supporting method of supporting concentration of maple sap in production of a maple syrup liquid, the supporting method including: acquiring current sap information including current color information and a current sugar content of the maple sap; and outputting predicted color information indicating a color of the maple syrup liquid predicted based on the current sap information and a target sugar content of a concentrated liquid obtained by concentrating the maple sap.

The contents of the following Japanese patent application and International application are incorporated herein by reference:
NO. 2020-113514 filed in JP on Jun. 30, 2020

### BACKGROUND

### 1. Technical Field

The present invention relates to a supporting method, a producing method, a computer readable storage medium, and a supporting device.

### 2. Related Art

Conventionally, a system and a method for producing maple syrup by concentrating maple sap have been known (see patent document 1, for example). Patent Document 1: U.S. Pat. No. 9,622,505

### SUMMARY

Maple sap is a product of nature with variable quality. Thus, it is difficult to predict the quality of the product after the concentration.

### BRIEF DESCRIPTION OF THE DRAWINGS

FIG. 1A illustrates an example of a configuration of a concentrating device 200.

FIG. 1B illustrates an example of a configuration of the concentrating device 200.

FIG. 2A illustrates an example of a configuration of a supporting device 100.

FIG. 2B illustrates a specific example of input information 52 and output information 53.

FIG. 3A is a block diagram illustrating an example of a supporting method performed by the supporting device 100.

FIG. 3B is a block diagram illustrating an example of a supporting method performed by the supporting device 100.

FIG. 3C is a block diagram illustrating an example of a supporting method performed by the supporting device 100.

FIG. 4 is a flowchart illustrating an example of a supporting method performed by the supporting device 100.

FIG. 5 is a flowchart illustrating an example of a producing method for a maple syrup liquid.

FIG. 6A illustrates an example of a configuration of a supporting device 300.

FIG. 6B illustrates a specific example of input information 52, output information 353, and current sap information 354.

FIG. 7A illustrates an example of a display section 330.

FIG. 7B illustrates an example of the display section 330.

FIG. 7C illustrates an example of the display section 330.

FIG. 8 is a flowchart illustrating an example of a supporting method performed by the supporting device 300.

FIG. 9 is a flowchart illustrating an example of a producing method for a maple syrup liquid.

FIG. 10 illustrates an example of a computer 2200 with which some or all of a plurality of aspects of the present invention may be embodied.

### DESCRIPTION OF EXEMPLARY EMBODIMENTS

Hereinafter, the present invention will be described through embodiments of the invention, but the following embodiments do not limit the claimed invention. Some combinations of features described in the embodiments may be unnecessary for solving means of the invention.

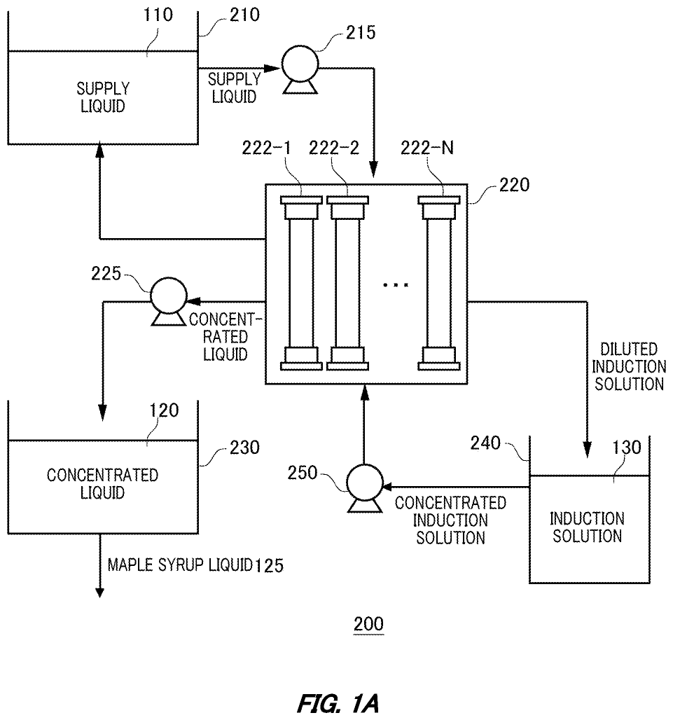

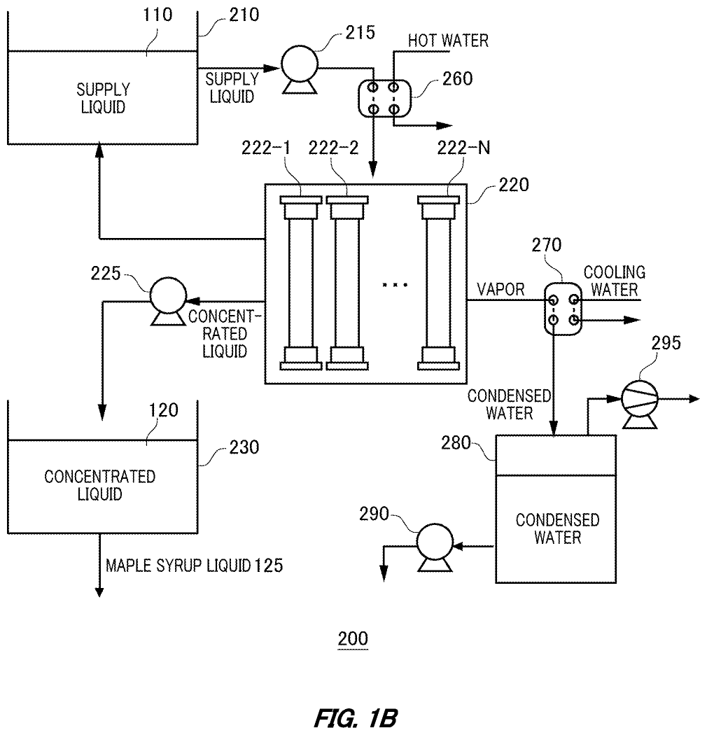

FIG. 1A illustrates an example of a configuration of a concentrating device 200. The concentrating device 200 of this example is an example of a concentrating device for concentrating maple sap before being concentrated, in the production of a maple syrup liquid. Maple sap harvested is concentrated by the concentrating device 200 of this example, and then is further concentrated by heating, for example. As a result, maple syrup with a predetermined sugar content is obtained as a product.

The concentrating device 200 includes a supply liquid tank 210, a concentrating section 220, a concentrated liquid tank 230, an induction solution tank 240, and an induction solution supply pump 250. The supply liquid tank 210 stores a supply liquid 110 which is maple sap. The supply liquid tank 210 may send the supply liquid 110 to the concentrating section 220, by means of a pump 215. The flow velocity (linear velocity) of the supply liquid 110 in the concentrating section 220 is affected by the pumping power of the pump 215. The supply liquid tank 210 may include a sensor for acquiring information such as the color, sugar content, and stored amount of the supply liquid 110.

The concentrating section 220 separates water from the supply liquid 110, to produce a concentrated liquid 120 obtained by concentrating the supply liquid 110. The concentrating section 220 of this example uses a forward osmosis method to concentrate the supply liquid 110. The concentrating section 220 includes one or a plurality of concentrating modules 222. The concentrating section 220 of this example includes N concentrating modules 222 that are a concentrating module 222-1 to a concentrating module 222-N.

The concentrating module 222 includes a filter for separating water from the supply liquid 110. The filter of the concentrating module 222 of this example is a forward osmosis membrane of a hollow fiber form. The supply liquid 110 is supplied into the hollow portion of the filter of the concentrating module 222.

In the concentrating module 222, an induction solution 130 supplied from the induction solution tank 240 flows on the outer side of the hollow fibers at a predetermined flow velocity. In the concentrating section 220, the supply liquid 110 comes into contact with the induction solution 130 via the filter of the concentrating module 222.

The concentrating section 220 may include a sensor for acquiring information such as the temperature, flow velocity, and concentration of the solution stored. The temperature of the solution may be referred to as concentration temperature. Maillard reaction occurs when the maple sap, which is the supply liquid 110, is heated, resulting in the color of the liquid gradually changing from nearly transparent to brown. Generally, a higher concentration temperature leads to an increase in the concentration speed of the supply liquid 110, resulting in a larger change in color.

The osmotic pressure of the induction solution 130 is higher than the osmotic pressure of the supply liquid 110. Thus, the water included in the supply liquid 110 moves toward the induction solution 130 through the filter of the concentrating module 222, with the difference in the osmotic pressure between the induction solution 130 and the supply liquid 110 serving as the driving force. The amount of this movement per unit time is referred to as flux and is expressed by L/m²h.

The flux determined by properties (such as the pore diameter) of the filter is referred to as initial flux, and the actual flux during operation of the concentrating device 200 is referred to as actual flux. For example, the actual flux is determined by the initial flux and a difference in concentration between the induction solution 130 and the supply liquid 110.

The concentrating section 220 may collect the concentrated liquid 120 generated inside the concentrating module 222, and supply the concentrated liquid 120 to the concentrated liquid tank 230 by means of the pump 225. The concentrated liquid 120 may be returned to the supply liquid tank 210. The concentrating section 220 may return, to the induction solution tank 240, the induction solution 130 mixed with the water separated from the supply liquid 110 to be diluted.

The concentrated liquid tank 230 stores the concentrated liquid 120 sent from the concentrating section 220. The concentrated liquid tank 230 may include a sensor for acquiring information such as the color, sugar content, and stored amount of the concentrated liquid 120.

The induction solution tank 240 stores the induction solution 130. For example, the induction solution 130 is an aqueous solution including MgCl₂. The induction solution tank 240 may include a sensor for measuring the concentration of the induction solution 130. The induction solution tank 240 may include an induction solution concentrating device that concentrates the diluted induction solution 130 by means of evaporation or the like.

In this manner, the induction solution tank 240 maintains the concentration of the induction solution 130 to be supplied to the concentrating section 220, at a concentration set in advance. The concentration of the induction solution 130 supplied to the concentrating section 220 is set to be higher than the concentration of the supply liquid 110.

The induction solution supply pump 250 supplies the induction solution 130 from the induction solution tank 240 to the concentrating section 220. The flow velocity of the induction solution 130 in the concentrating section 220 is affected by the pumping power of the induction solution supply pump 250.

The concentrating device 200 may further include a preprocessing concentrating section provided between the supply liquid tank 210 and the concentrating section 220. The preprocessing concentrating section may concentrate the supply liquid 110 to a certain sugar content by means of a reverse osmosis membrane module or the like, and supply the supply liquid 110 thus concentrated to the concentrating section 220. The concentrated liquid 120 is concentrated to the predetermined sugar content, which is 66 to 67%, by distillation or the like, whereby a maple syrup liquid 125 is produced.

FIG. 1B illustrates an example of a configuration of the concentrating device 200. In the example illustrated in FIG. 1B, the concentrating section 220 concentrates the supply liquid by a membrane distillation (MD) method. In FIG. 1B, elements that are commonly illustrated in FIG. 1A are denoted with the same reference numerals, and the description thereof will be omitted.

The concentrating device 200 includes the supply liquid tank 210, the concentrating section 220, the concentrated liquid tank 230, a heating section 260, a cooling section 270, a condensed water tank 280, an extraction pump 290, and a vacuum pump 295. The supply liquid 110 stored in the supply liquid tank 210 is sent to the concentrated liquid tank 230 by the pump 215, and passes through the heating section 260 before reaching the concentrated liquid tank 230. The supply liquid 110 is heated up to 40 to 50° C. by hot water flowing in the heating section 260.

The concentrating section 220 includes one or a plurality of concentrating modules 222. The filter of the concentrating module 222 is a hydrophobic porous dry membrane (MD membrane) of a hollow fiber form. The surface of the MD membrane may be coated with a water repellent. The actual flux is determined by a difference in vapor pressure between the initial flux and the porous membrane.

The supply liquid 110 is supplied into the hollow portion of the filter of the concentrating module 222. The water included in the supply liquid 110 turns into vapor and passes through the filter of the concentrating module 222, with the difference in vapor pressure between the supply liquid 110 and the porous membrane serving as the driving force. The vapor that has passed through the filter of the concentrating module 222 is decompressed and cooled by cooling water flowing in the cooling section 270 to be condensed. The condensed water is sent to the condensed water tank 280 maintained in a vacuum state by the vacuum pump 295, and is extracted by the extraction pump 290.

The concentrating section 220 may collect the concentrated liquid 120 generated inside the concentrating module 222 and supply the concentrated liquid 120 to the concentrated liquid tank 230 by means of the pump 225. Alternatively, the concentrated liquid 120 may be returned to the supply liquid tank 210 by means of a circulation pump, to be mixed with the supply liquid 110.

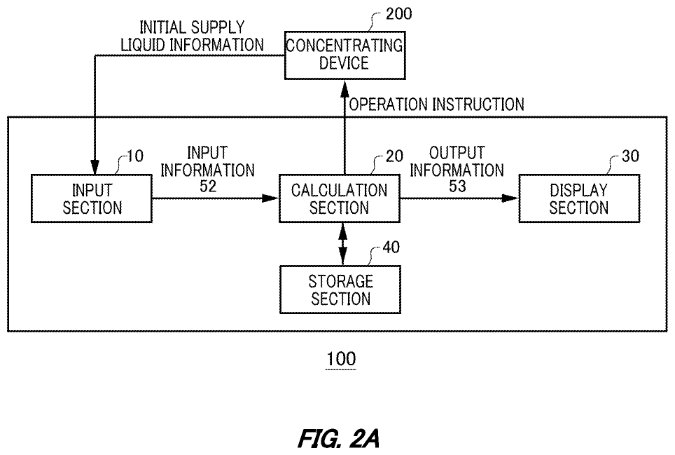

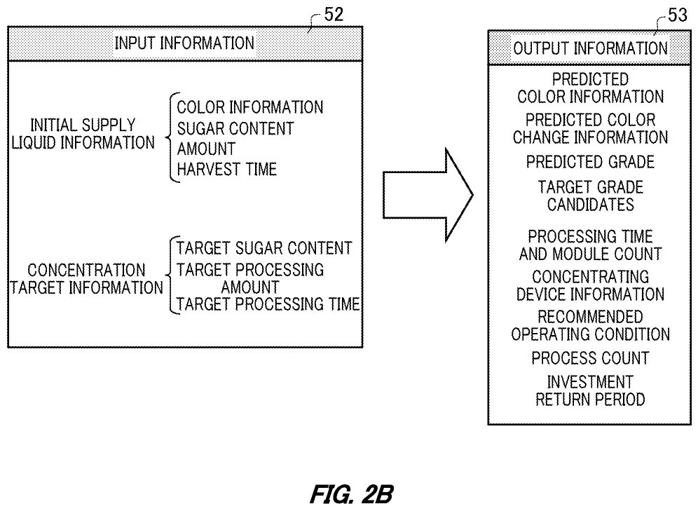

FIG. 2A illustrates an example of a configuration of a supporting device 100. The supporting device 100 includes an input section 10, a calculation section 20, a display section 30, and a storage section 40.

Here, the grade of the maple syrup liquid 125 is mainly determined by the color of the maple syrup liquid 125. For example, a more transparent color of the maple syrup liquid 125 indicates a higher grade of the maple syrup liquid 125, and a darker brown color of the maple syrup liquid 125 indicates a lower grade.

As described above, the maple sap in the supply liquid 110 has the color changing from nearly transparent to brown, upon being heated. Furthermore, the color of the concentrated liquid 120 changes to be darker by distillation after the concentration. Thus, to obtain the maple syrup liquid 125 of a higher grade, it is important to concentrate the supply liquid 110 while maintaining the concentrated liquid 120 in an initial state with a nearly transparent color.

The supporting device 100 calculates various conditions required for the concentrating device 200 to obtain the maple syrup liquid 125 of a desired grade, to support the production of the maple syrup liquid through concentration of maple sap. The supporting device 100 may be in wired or wireless connection with the concentrating device 200.

The input section 10 inputs input information 52 including initial supply liquid information and concentration target information. The initial supply liquid information is information on the supply liquid 110 that is maple sap before concentration. The concentration target information is information on the concentrated liquid 120 that is the target. The input of the input information 52 by the input section 10 may be implemented with an input device such as a keyboard, or through communications with a server. Alternatively, when the supporting device 100 is in wired or wireless connection with the concentrating device 200, the input section 10 may periodically acquire the initial supply liquid information from a sensor provided to the supply liquid tank 210 of the concentrating device 200.

The calculation section 20 outputs predicted color information indicating the color of the maple syrup liquid 125 predicted based on the input information 52, and calculates the condition of the concentrating device 200 required for obtaining the maple syrup liquid 125 corresponding to the predicted color information. The calculation section 20 outputs, as output information 53, the predicted information on the maple syrup liquid 125 and the condition of the concentrating device 200, to the display section 30.

The display section 30 displays the output information 53 acquired from the calculation section 20. For example, the display section 30 displays the output information 53 on a display. The display section 30 may transmit data as the output information 53 to the outside of the supporting device 100.

The storage section 40 stores information required for the calculation by the calculation section 20. The storage section 40 of this example stores information such as the specification of the concentrating module 222, cost information, and the product unit price of the maple syrup. The specification of the concentrating module 222 includes, for example, the pore diameter of the concentrating module 222. The cost information includes information on the running cost of the concentrating device 200, examples of which include chemical cleaning concentration, chemical purchased amount, chemical unit price, or electricity unit price. The product unit price of the maple syrup may be a product unit price determined by an industry group or the like for each grade. The storage section 40 may further store the information on the supply liquid 110, the information on the maple syrup liquid 125, and the condition of the concentrating device 200 used in the past, in association with each other.

The calculation section 20 may use a formula or a model obtained by simulation, machine learning, and combination thereof, to output the output information 53. As an example, the calculation section 20 uses ridge regression, PLS regression, random forest, SVR, or the like as a regression analysis and uses a genetic algorithm as machine learning. The calculation section 20 may use a formula or a model obtained by simulation, machine learning, and a combination thereof, to output the output information 53 based on the input information 52.

FIG. 2B illustrates a specific example of the input information 52 and the output information 53. The input information 52 includes the initial supply liquid information and the concentration target information. As an example, the initial supply liquid information includes color information and sugar content of the supply liquid 110. The color information of the supply liquid 110 may be a light transmittance measured by a spectrophotometer or a color-difference meter. Generally, a higher light transmittance of the supply liquid 110 corresponds to a color nearer to transparent, and a lower light transmittance of the supply liquid 110 corresponds to a darker brown color. Alternatively, the color information on the supply liquid 110 may be information as a result of converting the color of the supply liquid 110, identified from an image, into a color code. The sugar content of the supply liquid 110 may be measured by a saccharimeter, and the general sugar content (for example, 2.0%) of the maple sap before the concentration may be used as a default value.

The initial supply liquid information may further include the amount of the supply liquid 110. The amount of the supply liquid 110 affects a target processing time. The initial supply liquid information may further include the harvest time of the supply liquid 110. The harvest time affects a change in the color of the maple sap before the concentration.

As an example, the concentration target information includes the target sugar content of the concentrated liquid 120. For example, the target sugar content of the concentrated liquid 120 is 30% to 60%. The target sugar content of the concentrated liquid 120 may be 65%. The concentration target information may further include any one of the target processing amount and the target processing time. The target processing amount is a target amount of water to be separated from the supply liquid 110, and the target processing time is a time it takes for the concentrating section 220 to process the target processing amount.

The output information 53 includes predicted color information on the maple syrup liquid 125. For example, the predicted color information on the maple syrup liquid 125 is information with a light transmittance or a color code indicating the predicted color of the maple syrup liquid 125. The output information 53 may further include at least any of predicted color change information, predicted grade, target grade candidates, processing time and module count, concentrating device information, recommended operating condition, the process count, and investment return period.

The predicted color change information is information indicating prediction of the predicted change in the color of the maple syrup liquid 125 over time. The predicted grade is a predicted grade of the maple syrup liquid 125 corresponding to the predicted color information. The grade affects the product unit price.

The target grade candidates are a plurality of grades of the producible maple syrup liquid 125 in accordance with the predicted color information, that is, a plurality of grades of the maple syrup liquid 125 that can be set to be the target. Examples of the target grade candidates include golden, amber, dark, and very dark.

The processing time and module count are the processing time and the number of concentrating modules 222 required. The concentrating device information is information indicating the layout of the concentrating device 200 with a required size corresponding to the processing time and the number of concentrating modules 222. The process count is the number of processes corresponding to the operating time of the concentrating device 200. The investment return period is a period required for recovering the facility cost invested into the concentrating device 200.

The recommended operating condition is an operating condition recommended for obtaining the concentrated liquid 120 with the target sugar content from the supply liquid 110. The operating condition includes at least any of the linear velocity and concentration temperature of the supply liquid 110.

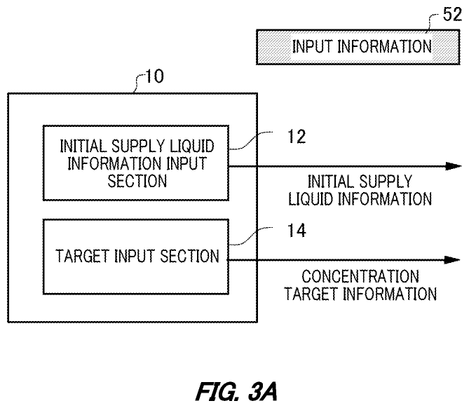

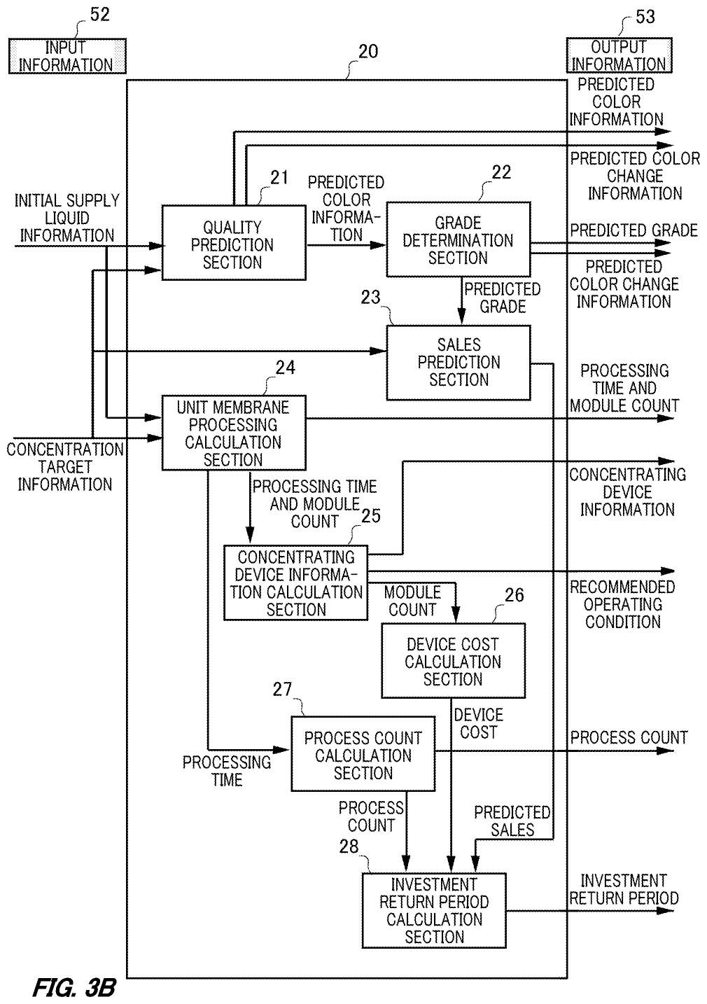

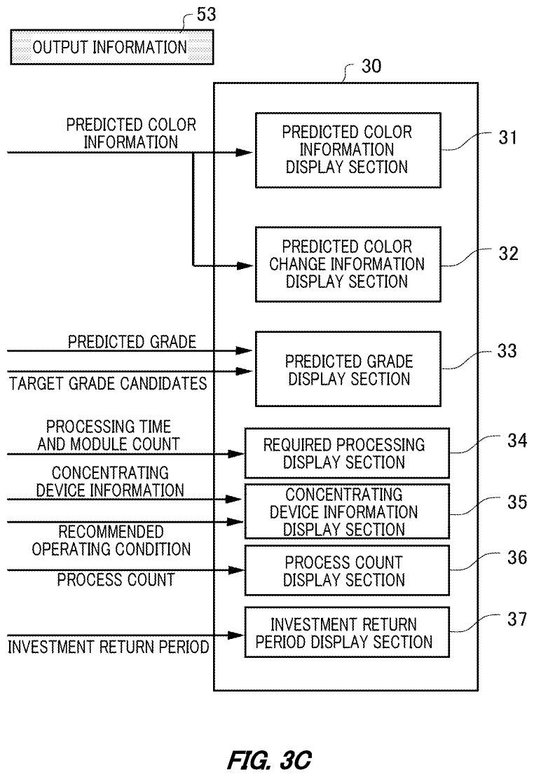

FIG. 3A is a block diagram illustrating an example of a supporting method performed by the supporting device 100. FIG. 3A mainly illustrates the input section 10. The input section 10 includes an initial supply liquid information input section 12 and a target input section 14. The initial supply liquid information input section 12 inputs the initial supply liquid information. The target input section 14 inputs the concentration target information. The input section 10 outputs, as the input information 52 to the calculation section 20, the initial supply liquid information and concentration target information input.

FIG. 3B is a block diagram illustrating an example of a supporting method performed by the supporting device 100. FIG. 3B mainly illustrates the calculation section 20. The calculation section 20 includes a quality prediction section 21, a grade determination section 22, a sales prediction section 23, a unit membrane processing calculation section 24, a concentrating device information calculation section 25, a device cost calculation section 26, a process count calculation section 27, and an investment return period calculation section 28.

The quality prediction section 21 predicts the color of the maple syrup liquid 125 based on the initial supply liquid information and the concentration target information, and outputs predicted color information indicating the predicted color of the maple syrup liquid 125. The quality prediction section 21 outputs the predicted color information to the grade determination section 22, and to the display section 30.

The quality prediction section 21 creates a learning model using the sugar content and color information of the supply liquid 110, the operating condition (the linear velocity and concentration temperature of the supply liquid 110) of the concentrating device 200, and the predicted color information obtained. The sugar content and color information of the supply liquid 110 refer to the sugar content and color information of the supply liquid 110 before the concentration.

For example, the quality prediction section 21 uses the sugar content and color information, the linear velocity, the processing amount, and the concentration temperature of the supply liquid 110, as well as the light transmittance or color code of the maple syrup liquid 125 as training data to train the learning model so as to predict the light transmittance or color code of the maple syrup liquid 125. Alternatively, a trained model may be implemented in the quality prediction section 21.

The quality prediction section 21 may further output the predicted color change information based on the initial supply liquid information and the predicted color information when the initial supply liquid information includes the harvest time. The quality prediction section 21 predicts a change in the color information of the supply liquid 110 over time from the color information and the harvest time of the supply liquid 110, and outputs the predicted color change information indicating a change in the predicted color information of the maple syrup liquid 125 corresponding to this change over time. The change over time is, for example, a change over a month or three months.

The grade determination section 22 outputs the predicted grade of the maple syrup liquid 125 based on the predicted color information acquired from the quality prediction section 21. For example, the grade determination section 22 compares the predicted color information on the maple syrup liquid 125 with a grade determined in advance for each light transmittance, and outputs the grade corresponding to the predicted color information as the predicted grade. Alternatively, the grade determination section 22 may compare the predicted color information on the maple syrup liquid 125 with a grade determined in advance for each color code, and output the grade corresponding to the predicted color information as the predicted grade. The grade determination section 22 outputs the predicted color information on the maple syrup liquid 125 to the sales prediction section 23 and to the display section 30.

Furthermore, the grade determination section 22 may output the target grade candidates of the producible maple syrup liquid 125 in accordance with the predicted color information. The grade determination section 22 may output the target grade candidates to the display section 30.

The sales prediction section 23 predicts the sales of a product obtained from the maple syrup liquid 125, based on the initial supply liquid information and the concentration target information and on the predicted grade acquired from the grade determination section 22. For example, the sales prediction section 23 calculates the predicted sales of the product obtained from the maple syrup liquid 125, from the amount of the supply liquid 110, the target processing amount, the predicted grade of the maple syrup liquid 125, and a product unit price for each grade determined in advance.

The sales prediction section 23 may use the grade as a result of converting the predicted grade of the maple syrup liquid 125 using a predetermined coefficient, as the grade of the product. The sales prediction section 23 outputs to the investment return period calculation section 28, predicted sales information indicating the predicted sales thus calculated. The sales prediction section 23 may also output the predicted sales information to the display section 30.

Based on the initial supply liquid information and the concentration target information, the unit membrane processing calculation section 24 calculates the per unit time processing amount of one concentrating module 222, and calculates at least any of the processing time and the number of concentrating modules 222 required, from the per unit time processing amount thus calculated. The processing amount is the amount of water separated from the supply liquid 110. The unit membrane processing calculation section 24 outputs information indicating the processing time and the module count calculated to the concentrating device information calculation section 25, and outputs information indicating the module count to the process count calculation section 27. Furthermore, the unit membrane processing calculation section 24 outputs the information indicating the processing time and the module count to the display section 30.

The concentrating device information calculation section 25 calculates the required size of the concentrating device 200 based on the information indicating the processing time and the module count acquired from the unit membrane processing calculation section 24, and outputs the concentrating device information indicating the layout of the concentrating device 200 corresponding to the size thus calculated, to the display section 30. The concentrating device information calculation section 25 outputs the information indicating the number of concentrating modules 222 to the device cost calculation section 26.

Furthermore, the concentrating device information calculation section 25 outputs the recommended operating condition based on the initial supply liquid information and the concentration target information. The operating condition includes at least any of the linear velocity and the concentration temperature of the supply liquid 110. The recommended operating condition is an operating condition recommended for obtaining the concentrated liquid 120 with the target sugar content from the supply liquid 110.

The concentrating device information calculation section 25 may output the recommended operating condition further based on the size of the concentrating device 200 calculated. The concentrating device information calculation section 25 may acquire the information on the supply liquid 110, the information on the maple syrup liquid 125, and the condition of the concentrating device 200 used in the past from the storage section 40, and output the recommended operating condition further based on these pieces of information. There may be one or a plurality of recommended operating conditions.

The concentrating device information calculation section 25 may acquire the recommended operating condition by repeatedly using a model using a genetic algorithm. This model may be, for example, trained to be capable of predicting the recommended operating condition (the linear velocity and the concentration temperature of the supply liquid 110), by using as the training data, the sugar content and color information of the supply liquid 110, the processing amount, the target processing time, and the operating condition (linear velocity and concentration temperature of the supply liquid 110). The sugar content and the color information of the supply liquid 110 refer to the sugar content and the color information of the supply liquid 110 before concentration. After a plurality of recommended operating conditions are acquired, any of the selection, crossover, and mutation may be performed on these recommended operating conditions to generate a plurality of next generation recommended operating conditions.

As described later, upon acquiring a user input indicating the target grade selected from the target grade candidates from the display section 30, the concentrating device information calculation section 25 calculates the recommended operating condition further based on the user input. The concentrating device information calculation section 25 outputs the recommended operating condition to the display section 30.

The device cost calculation section 26 calculates device cost information indicating the installation cost of the concentrating device 200, based on the required number of concentrating modules 222 acquired from the concentrating device information calculation section 25, the predetermined unit price of the concentrating module 222, and the like. The device cost calculation section 26 outputs the calculated device cost information to the investment return period calculation section 28.

The process count calculation section 27 calculates the number of processes required for generating the concentrated liquid 120, based on the information indicating the processing time acquired from the unit membrane processing calculation section 24. The process count calculation section 27 outputs the information indicating the number of processes calculated, to the investment return period calculation section 28.

The investment return period calculation section 28 calculates the investment return period based on the information indicating the predicted sales information, the device cost information, and the process count acquired. The investment return period calculation section 28 outputs the calculated investment return period to the display section 30.

In this manner, the calculation section 20 outputs, as the output information 53 to the display section 30, information obtained by the calculation such as the predicted color information, the predicted color change information, the predicted grade, the processing time and the module count, the concentrating device information, the operating condition, the process count, and the investment return period. The calculation section 20 may store, in the storage section 40, information obtained by the calculation such as the predicted color information, the predicted color change information, the predicted grade, the processing time and the module count, the concentrating device information, the operating condition, the process count, and the investment return period.

FIG. 3C is a block diagram illustrating an example of a supporting method performed by the supporting device 100. FIG. 3C mainly illustrates the display section 30. The display section 30 includes a predicted color information display section 31, a predicted color change information display section 32, a predicted grade display section 33, a required processing display section 34, a concentrating device information display section 35, a process count display section 36, and an investment return period display section 37, respectively displaying the predicted color information, the predicted color change information, the predicted grade, the processing time and the module count, the concentrating device information, the process count, and the investment return period acquired from the calculation section 20.

The predicted color information display section 31 and the predicted color change information display section 32 may display the color code, indicated by the predicted color information and the predicted color change information acquired, as text information, or may convert the color code into an image and display the image. The predicted grade display section 33 displays the predicted grade acquired.

Furthermore, the predicted grade display section 33 may display the target grade candidates. The predicted grade display section 33 may receive a user input indicating a target grade selected from the target grade candidates. The predicted grade display section 33 transmits the received user input to the calculation section 20. When the calculation section 20 acquires the user input, the concentrating device information calculation section 25 calculates the recommended operating condition further based on the user input as described above.

The required processing display section 34 may display the possible number of concentrating modules 222 and the processing time corresponding to each of the module counts, in association with each other. The concentrating device information display section 35 may display candidates of the device layout in accordance with the display content of the required processing display section 34. The concentrating device information display section 35 may further display the recommended operating condition, and receive a user input indicating the operating condition selected by the user from the recommended operating condition. Upon acquiring a plurality of recommended operating conditions, the concentrating device information display section 35 may display these recommended operating conditions. The concentrating device information display section 35 may display a plurality of recommended operating conditions acquired in the descending order of the target achieving probability.

The display section 30 may display all or a part of the predicted color information display section 31, the predicted color change information display section 32, the predicted grade display section 33, the required processing display section 34, the concentrating device information display section 35, the process count display section 36, and the investment return period display section 37. The predicted color information display section 31 and the predicted color change information display section 32 may display all or a part of the predicted color information as well as the text information and the converted image indicating the color code. The required processing display section 34 may display one or both of the possible number and processing time of the concentrating modules 222.

The calculation section 20 supplies the recommended operating condition to the concentrating device 200 to cause the concentration of the supply liquid 110 under the operating condition indicated by the recommended operating condition. Upon acquiring a plurality of recommended operating conditions, the calculation section 20 may output, in accordance with a user input, any one of the recommended operating conditions selected by the user to the concentrating device 200.

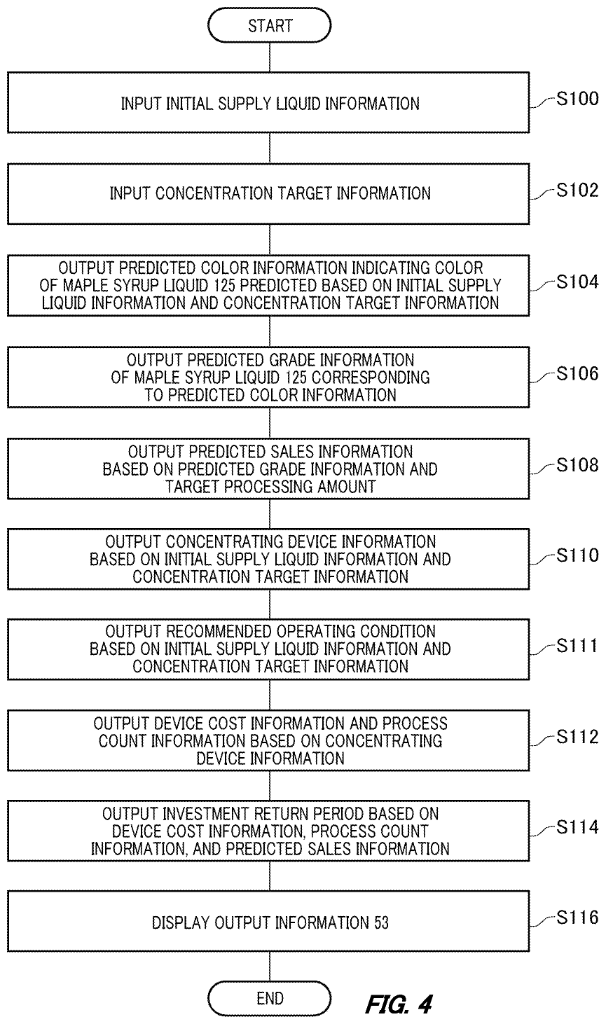

FIG. 4 is a flowchart illustrating an example of a supporting method performed by the supporting device 100. In step S100, the initial supply liquid information including the sugar content and color information of the supply liquid 110 that is maple sap before concentration is input. In step S100, the harvest time may be further input as the initial supply liquid information. In step S102, the concentration target information including the target sugar content of the concentrated liquid 120 obtained by concentrating the maple sap is input. In step S104, the predicted color information indicating the color of the maple syrup liquid 125 predicted based on the initial supply liquid information and the concentration target information is output.

In step S106, the predicted grade information of the maple syrup liquid 125 corresponding to the predicted color information is output. In step S108, the predicted sales information is output based on the predicted grade information and the target processing amount.

In step S110, the concentrating device information including the required number of concentrating modules is output based on the initial supply liquid information and the concentration target information.

In step S111, the recommended operating condition is output based on the initial supply liquid information and the concentration target information.

In step S112, the device cost information and the process count information are output based on the concentrating device information.

In step S114, the investment return period is output based on the device cost information, the process count information, and the predicted sales information.

In step S116, the output information 53 including at least any of the predicted color information, the predicted color change information, the predicted grade, the processing time and the module count, the concentrating device information, the operating condition, the process count, and the investment return period is displayed.

As described above, in the supporting method performed by the supporting device 100 of this example, the predicted color information on the maple syrup liquid 125 is output as the output information 53, so that the color of the maple syrup liquid 125, that is, the grade of the maple syrup liquid 125 can be predicted and displayed regardless of the variation of the quality of the supply liquid 110. Furthermore, in the supporting method performed by the supporting device 100 of this example, information related to the device required for obtaining the maple syrup liquid 125 of the predicted grade is output as the output information 53, whereby the production of the maple syrup liquid using the concentrating device 200 can be supported.

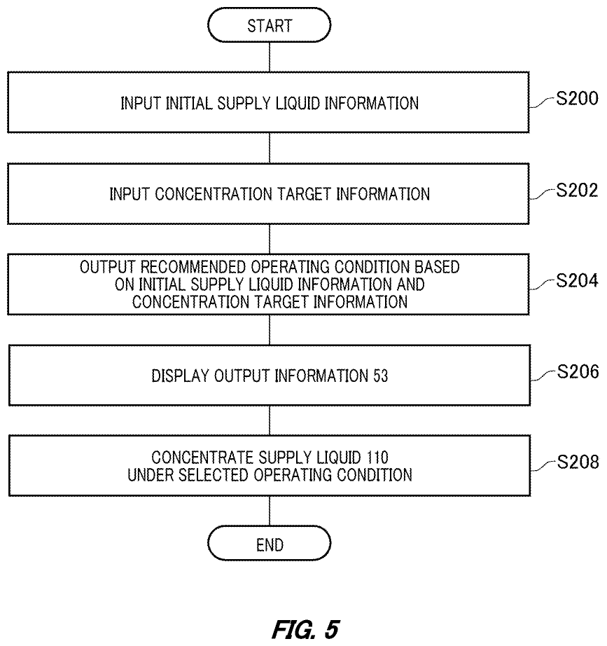

FIG. 5 is a flowchart illustrating an example of a producing method for a maple syrup liquid. In FIG. 1A to FIG. 4, a description is given on the supporting method for supporting the production of the maple syrup liquid using the supporting device 100 prior to the introduction of the concentrating device 200. In FIG. 5, a producing method for a maple syrup liquid performed using the supporting device 100 while the concentrating device 200 is operating will be described.

In step S200, the supporting device 100 inputs the initial supply liquid information including the sugar content and color information of the supply liquid 110 that is maple sap before concentration. In step S202, the concentration target information including the target sugar content of the concentrated liquid 120 obtained by concentrating the maple sap is input.

In step S204, the supporting device 100 outputs the recommended operating condition based on the initial supply liquid information and the concentration target information. The supporting device 100 may output the predicted color information indicating the color of the maple syrup liquid 125 predicted based on the initial supply liquid information and the concentration target information. Furthermore, the supporting device 100 may output the target grade candidates of the producible maple syrup liquid 125 in accordance with the predicted color information.

In step S206, the supporting device 100 displays the output information 53. The output information 53 at least includes the recommended operating condition. The supporting device 100 may receive a user input indicating the operating condition selected from the recommended operating condition by the user. The supporting device 100 may output an operation instruction, for concentrating the supply liquid 110 under the selected operating condition, to the concentrating device 200.

The output information 53 may include target grade candidates. The supporting device 100 may receive a user input indicating the grade selected from the target grade candidates by the user. When the user input is received, the processing returns to step S204, where the supporting device 100 may output the recommended operating condition further based on the user input received.

In step S208, the concentrating device 200 concentrates the supply liquid 110 under the selected operating condition.

As described above, in the producing method for the maple syrup liquid of this example, the supporting device 100 outputs the recommended operating condition based on the initial supply liquid information and the concentration target information, and the concentrating device 200 concentrates the supply liquid 110 under the operating condition selected from the recommended operating condition. Thus, the maple syrup liquid 125 with the target sugar content and color can be produced, regardless of the variation of the quality of the supply liquid 110. In conventional cases, the operating condition has not been optimized, and thus a maple syrup liquid of a low grade may be produced. On the other hand, with the producing method for a maple syrup liquid of this example, a maple syrup liquid of a higher grade can be produced.

Furthermore, in the producing method for a maple syrup liquid of this example, the predicted color information is output, and the target grade candidates of the producible maple syrup liquid 125 are output in accordance with the predicted color information, and the recommended operating condition is output based on the target grade selected by the user. With this configuration, the supply liquid 110 is concentrated under the optimum operating condition, so that desired grades can be selectively obtained.

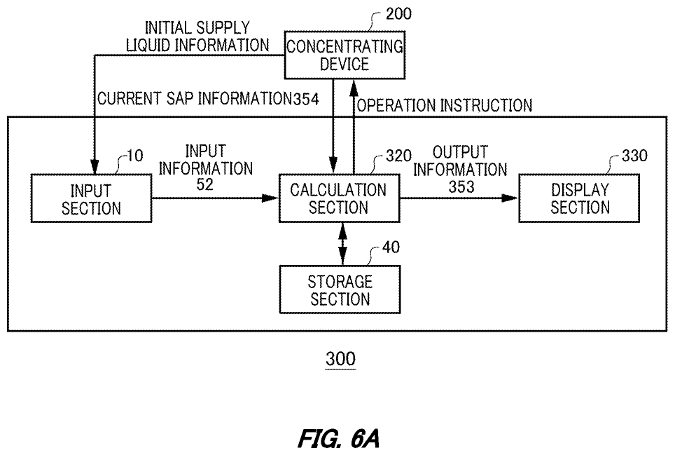

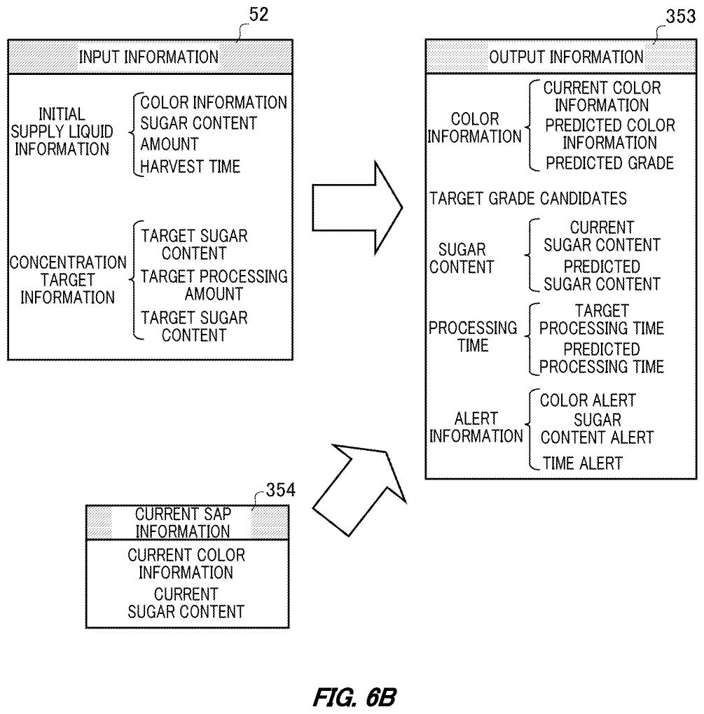

FIG. 6A illustrates an example of a configuration of a supporting device 300. Here, components that are the same as those in the supporting device 100 are denoted by the same reference numerals, and the description thereof will be omitted.

The supporting device 300 includes the input section 10, a calculation section 320, a display section 330, and the storage section 40. The supporting device 300 may be in wired or wireless connection with the concentrating device 200. The calculation section 320 acquires the input information 52 from the input section 10, and also acquires current sap information 354 from the concentrating device 200. As in the case of the supporting device 100, the input section 10 may periodically acquire the initial supply liquid information from a sensor provided to the supply liquid tank 210 of the concentrating device 200. The supporting device 300 outputs predicted information based on the current sap information 354 acquired, to support the concentration of the maple sap in the production of a maple syrup liquid.

The current sap information 354 is information indicating the current state of the supply liquid 110 being concentrated in the concentrating section 220, and includes current color information and current sugar content. The calculation section 320 may acquire the current sap information 354 from the concentrating device 200 at a predetermined interval. The calculation section 320 outputs the acquired current sap information 354 to the display section 330, as output information 353.

The calculation section 320 outputs the predicted color information indicating the predicted color of the maple syrup liquid 125, based on the input information 52 and the current sap information 354. The color of the maple syrup liquid 125 is one of the factors based on which the grade of the maple syrup liquid 125 is determined.

For example, the calculation section 320 calculates a sugar content difference between the current sugar content and the target sugar content of the supply liquid 110, and calculates the predicted color information based on an impact of the sugar content difference on a change in the color. The calculation section 320 outputs the calculated predicted color information to the display section 330, as the output information 353.

The calculation section 320 may use the learning model to acquire the predicted color information, as with the calculation section 20 of the supporting device 100. The calculation section 320 uses the sugar content and color information, the linear velocity, the processing amount, the concentration temperature of the supply liquid 110, as well as the light transmittance or color code of the maple syrup liquid 125 as training data to train the learning model so as to predict the light transmittance or color code of the maple syrup liquid 125. It should be noted that, while the calculation section 20 of the supporting device 100 uses the sugar content and color information of the supply liquid 110 before the concentration as the sugar content and color information of the supply liquid 110 in the training data, the calculation section 320 of this example uses the current sugar content and color information of the supply liquid 110.

The calculation section 320 outputs the predicted grade information as a result of predicting the grade of the maple syrup liquid 125 based on the predicted color information. The calculation of the predicted grade is the same as that by the calculation section 20 of the supporting device 100, and thus the description will be omitted.

The calculation section 320 outputs the target grade candidates of the producible maple syrup liquid 125 based on the predicted color information. The target grade candidates are a plurality of grades that can be set as targets. The calculation section 320 calculates the operating condition of the concentrating device 200 based on a user input indicating a target grade selected from the target grade candidates, and outputs the operating condition change information for changing the operating condition. The operating condition change information may be information indicating the operating condition with which the predicted grade matches the target grade. The calculation section 320 may output the operating condition change information to the concentrating device 200 to make the concentrating device 200 operate under the operating condition indicated by the operating condition change information.

The calculation section 320 outputs the operating condition indicated by the operating condition change information, further based on the current sap information 354 and concentration target information. The calculation section 320 may acquire the operating condition using a learning model, as with the calculation section 20 of the supporting device 100. This model may be, for example, trained to be capable of outputting the operating condition with which the predicted grade matches the target grade, by using as training data, the sugar content and color information of the supply liquid 110, the processing amount, the target processing time, and the current operating condition, for example. It should be noted that, while the calculation section 20 of the supporting device 100 uses the sugar content and color information of the supply liquid 110 before the concentration as the sugar content and color information of the supply liquid 110 in the training data, the calculation section 320 of this example uses the current sugar content and color information of the supply liquid 110.

Furthermore, the calculation section 320 outputs the predicted sugar content indicating the sugar content of the concentrated liquid 120 predicted based on the current sugar content and the target processing time. The predicted sugar content is a sugar content of the concentrated liquid 120 predicted to be achieved when the target processing time elapses. For example, the calculation section 320 calculates the amount of sugar from the current sugar content of the supply liquid 110, and calculates the predicted processing amount from the per unit time processing amount of the concentrating section 220. The predicted processing amount is the amount of water separated from the supply liquid 110 from the current time to the target end time.

When the concentrating section 220 concentrates the supply liquid 110 by the forward osmosis method, the calculation section 320 may calculate the per unit time processing amount from the initial flux of the concentrating module 222, the flow velocity of the induction solution 130, and a concentration difference between the supply liquid 110 and the induction solution 130. The calculation section 320 may perform correction, machine learning, simulation, and the like on the initial flux, based on the operation history of the concentrating device 200 stored in the storage section 40. The calculation section 320 may calculate the predicted processing amount further based on the viscosity of the supply liquid 110.

When the concentrating section 220 uses a membrane distillation method to concentrate the supply liquid 110, the calculation section 320 may calculate the per unit time processing amount from the initial flux of the concentrating module 222, the flow velocity of the supply liquid 110, the temperature of the supply liquid 110, and the degree of vacuum outside the hollow fibers of the concentrating module 222. The calculation section 320 may perform the machine learning, simulation, and the like, based on the operation history of the concentrating device 200 stored in the storage section 40. The calculation section 320 may calculate the predicted processing amount further based on the viscosity of the supply liquid 110 or the pressure applied to the concentrating module 222.

The calculation section 320 outputs the predicted end time of the concentration based on the current sugar content of the supply liquid 110 and the predicted sugar content of the concentrated liquid 120. For example, the calculation section 320 calculates a correction time for correcting the target processing time based on a difference between the target sugar content and the predicted sugar content, and calculates a sum of the target processing time and the correction time as a predicted processing time. The calculation section 320 outputs as the output information 353 to the display section 330, the target end time corresponding to the target processing time and the predicted end time corresponding to the predicted processing time.

The calculation section 320 may output alert information as the output information 353 to the display section 330 when the predicted state of the maple syrup liquid 125 deviates from the target. The calculation section 320 may output color alert information when the predicted color of the maple syrup liquid 125 is outside a predetermined color range.

The calculation section 320 may output time alert information when a difference between the predicted end time and the target end time exceeds a predetermined time threshold. The calculation section 320 may output sugar content alert information when a difference between the predicted sugar content and the target sugar content exceeds a predetermined sugar content threshold. The display section 330 displays the time alert information and the sugar content alert information, to notify the user of a deviation of the predicted value from the target value.

The time threshold may at least include a first time threshold and a second time threshold greater than the first time threshold. The time alert information may include the operating condition change information when the difference between the predicted end time and the target end time exceeds the second time threshold. The operating condition change information may be information indicating an operating condition resulting in a difference between the predicted end time and the target end time not exceeding the second time threshold.

The sugar content threshold may at least include a first sugar content threshold and a second sugar content threshold greater than the first sugar content threshold. The sugar content alert information may include the operating condition change information when the difference between the predicted sugar content and the target sugar content exceeds the second sugar content threshold. The operating condition change information may be information indicating an operating condition resulting in a difference between the predicted sugar content and the target sugar content not exceeding the second sugar content threshold.

The calculation section 320 may output the operating condition change information based on the concentration target information and the current sap information 354. The operating condition indicated by the operating condition change information may be at least any of the concentration temperature and the linear velocity of the supply liquid 110. The display section 330 may receive a user input indicating that the user has permitted a change to the operating condition indicated by the operating condition change information. When the display section 330 receives the user input, the calculation section 320 may output the operating condition change information to the concentrating device 200 to make the concentrating device 200 operate under the operating condition indicated by the operating condition change information. The calculation of the operating condition is the same as in the case where the operating condition is calculated based on the user input indicating the target grade, and thus the description thereof will be omitted herein.

FIG. 6B illustrates a specific example of the input information 52, the output information 353, and the current sap information 354. Here, the input information 52 is the same as that in the case of the supporting device 100, and thus the description thereof will be omitted.

The current sap information 354 includes the current color information and the current sugar content of the supply liquid 110. For example, the current color information is information as a result of converting the color of the supply liquid 110, acquired from a light transmittance measurement device, a color-difference meter, a camera, or the like, into a color code. For example, the current sugar content is the sugar content of the supply liquid 110 acquired by the saccharimeter.

The output information 353 includes color information, target grade candidates, sugar content, processing time, and alert information. The color information may include current color information, predicted color information, and predicted grade. The sugar content may include the current sugar content of the supply liquid 110 and the predicted sugar content of the concentrated liquid 120. The processing time includes the target processing time and the predicted processing time. The alert information includes color alert information, sugar content alert information, and time alert information.

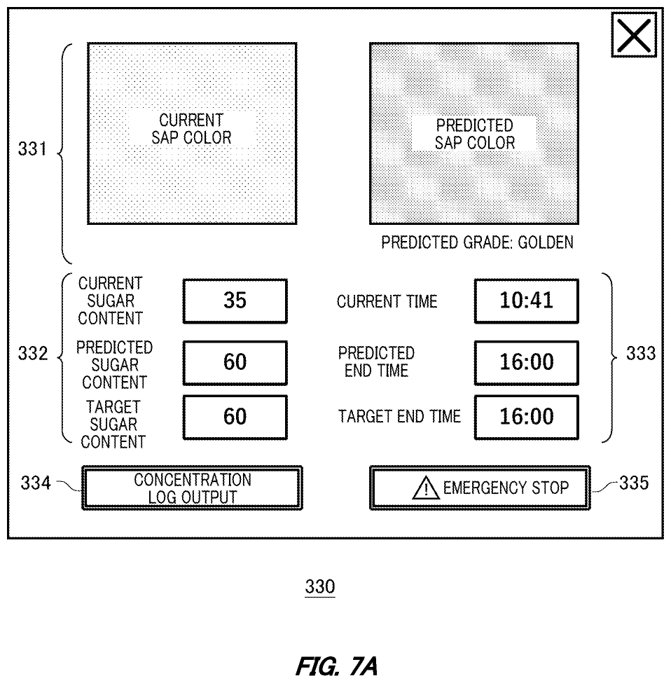

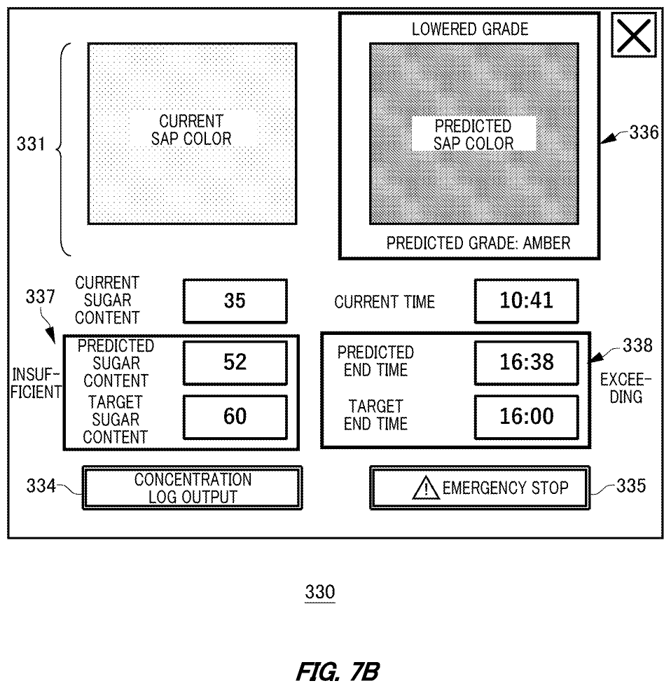

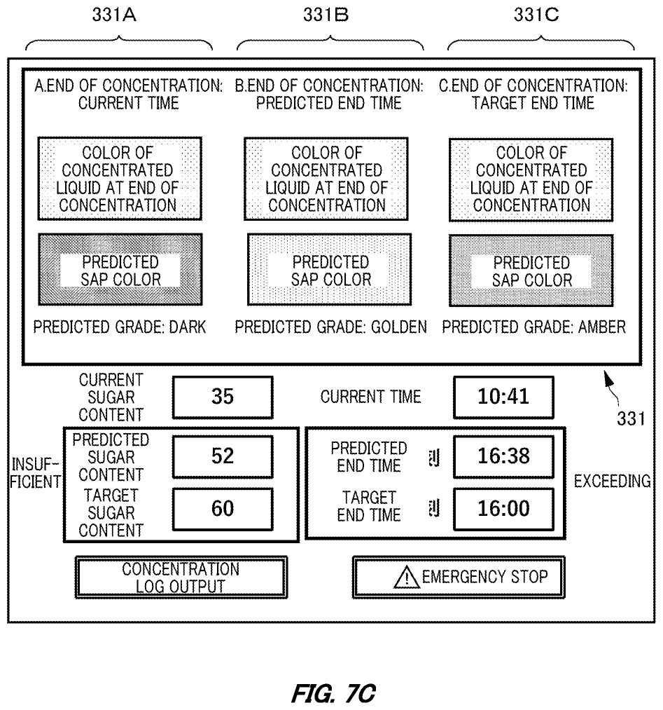

FIG. 7A illustrates an example of the display section 330. The display section 330 includes a color information display section 331 displaying color information, a sugar content display section 332 displaying information on the sugar content, and a time display section 333 displaying information on processing time.

FIG. 7A illustrates the display section 330 in a case where the difference between the predicted state of the maple syrup liquid 125 and the target is within a predetermined range. The example illustrated in FIG. 7A indicates that the current sugar content is 35%, the predicted sugar content is 60% at the target end time, which is 16:00, and the predicted end time is 16:00, which is the same as the target end time. FIG. 7A illustrates that the predicted color of the maple syrup liquid 125 is golden, which is the target sap color, when the concentration ends at 60%, which is the predicted sugar content, and then distillation is performed to achieve a predetermined sugar content 67%.

For example, the color information display section 331 displays the current sap color and the predicted sap color. Here, the current sap color is the color of the supply liquid 110 at the current time, and the predicted sap color is the predicted color of the maple syrup liquid 125 as a result of distillation to achieve the predetermined sugar content 67% after the concentration. Displaying the current sap color and the predicted sap color means that images as a result of converting the color codes corresponding to the current sap color and predicted sap color are displayed with respective texts “current sap color” and “predicted sap color”. The color information display section 331 may display the text indicating the predicted grade in association with the predicted sap color.

The color information display section 331 may further display the target grade candidates. The color information display section 331 may receive a user input indicating a target grade selected from the target grade candidates. When the color information display section 331 receives the user input, the calculation section 320 may calculate the operating condition based on the user input, and output the operating condition change information to the concentrating device 200 to make the concentrating device 200 operate under the operating condition indicated by the operating condition change information.

The sugar content display section 332 displays the current sugar content of the supply liquid 110, as well as the predicted sugar content and the target sugar content of the concentrated liquid 120. The time display section 333 may display a predicted processing time point and a target processing time point corresponding to the predicted processing time and the target processing time, together with the current time.

The display section 330 may further include a concentration log output button 334 for displaying the history of the current sap information 354 and an emergency stop button 335 for stopping the operation of the concentrating device 200. Upon detecting pressing of the concentration log output button 334, the calculation section 320 may acquire the history of the current sap information 354 from the storage section 40, and output the history to the display section 330. Upon detecting pressing of the emergency stop button 335, the calculation section 320 may output a signal instructing the concentrating device 200 to stop operating.

FIG. 7B illustrates an example of the display section 330. FIG. 7B illustrates the display section 330 in a case where the difference between the predicted state of the maple syrup liquid 125 and the target is outside the predetermined range. The example illustrated in FIG. 7B indicates that the current sugar content is 35%, the predicted sugar content is 52% at the target end time, which is 16:00, and the predicted end time is 16:38 when the concentration to achieve the target sugar content of 60% is performed. FIG. 7B illustrates that the predicted sap color is darker than golden, which is the target sap color, when the concentration ends at 52%, which is the predicted sugar content, and then distillation is performed to achieve a predetermined sugar content 67%.

The calculation section 320 may output a change instruction for changing the operating condition to the concentrating device 200, when the difference between the predicted state of the maple syrup liquid 125 and the target is outside the predetermined range. This predetermined range refers to a range not exceeding a predetermined threshold from a target value (target sugar content or target end time). The time threshold may at least include a first time threshold and a second time threshold greater than the first time threshold. The sugar content threshold may at least include a first sugar content threshold and a second sugar content threshold greater than the first sugar content threshold.

In this example, it is assumed that the first time threshold is set to 30 minutes, and the second time threshold is set to 60 minutes. Referring to FIG. 7B again, the difference between the predicted end time and the target end time exceeds 30 minutes, which is the first time threshold. The calculation section 320 may output to the display section 330, time alert information 338 for notifying the user of the deviation of the predicted value from the target value.

Furthermore, in this example, it is assumed that the first sugar content threshold is set to be 10%, and the second sugar content threshold is set to be 15%. Referring to FIG. 7B again, the difference between the predicted sugar content and the target sugar content exceeds 10%, which is the first sugar content threshold, (insufficient). The calculation section 320 may output to the display section 330, sugar content alert information 337 for notifying the user of the deviation of the predicted value from the target value.

Alternatively, in this example, it is assumed that the first time threshold is set to 20 minutes, and the second time threshold is set to 30 minutes. Referring to FIG. 7B again, the difference between the predicted end time and the target end time exceeds 30 minutes, which is the second time threshold. The calculation section 320 may further output the operating condition change information as the time alert information 338.

For example, the calculation section 320 outputs the operating condition change information indicating the concentration temperature and the linear velocity of the supply liquid 110 higher than those at the current time to the concentrating device 200, to increase the concentration speed in the concentrating section 220 to achieve an earlier predicted end time.

Furthermore, in this example, it is assumed that the first sugar content threshold is set to be 5%, and the second sugar content threshold is set to be 10%. Referring to FIG. 7B again, the difference between the predicted sugar content and the target sugar content exceeds 10%, which is the second sugar content threshold, (insufficient). The calculation section 320 may further output the operating condition change information as the sugar content alert information 337.

For example, the calculation section 320 outputs the operating condition change information indicating the concentration temperature and the linear velocity of the supply liquid 110 respectively lower and higher than those at the current time to the concentrating device 200, to increase the concentration speed in the concentrating section 220 while suppressing the color change of the supply liquid 110.

The display section 330 may display color alert information 336 overlapped on the predicted sap color, when the predicted color of the maple syrup liquid 125 is outside the predetermined color range. Furthermore, when the calculation section 320 outputs the sugar content alert information 337, the display section 330 may display the sugar content alert information 337 overlapped on the predicted sugar content and the target sugar content of the concentrated liquid 120. Furthermore, when the calculation section 320 outputs the time alert information 338, the display section 330 may display the time alert information 338 overlapped on the predicted processing time point and the target processing time point.

When the calculation section 320 outputs the operating condition change information, the display section 330 may display the operating condition change information. The display section 330 may receive a user input indicating that the user has permitted a change to the operating condition indicated by the operating condition change information. When the display section 330 receives the user input, the calculation section 320 may output the operating condition change information to the concentrating device 200 to make the concentrating device 200 operate under the operating condition indicated by the operating condition change information.

FIG. 7C illustrates an example of the display section 330. In FIG. 7C, an example is described in which the color information display section 331 includes a field for displaying a combination of the predicted color of the concentrated liquid 120 at the end of the concentration and the predicted sap color for each concentration end timing. Description on elements commonly illustrated in FIG. 7A and FIG. 7B will be omitted.

A field 331A displays a combination of the predicted color of the concentrated liquid 120 at the end of the concentration and the predicted sap color when the concentration ends at the current time point. Thus, the predicted color of the concentrated liquid 120 at the end of the concentration is the current sap color, which is the color of the supply liquid 110 at the current time, and the predicted sap color is the predicted color of the maple syrup liquid 125 as a result of distillation to achieve the predetermined sugar content 67% after the concentration.

The example illustrated in FIG. 7C indicates that the current sugar content is 35%, the predicted sugar content is 52% at the target end time, which is 16:00, and the predicted end time is 16:38 when the concentration to achieve the target sugar content of 60% is performed. Here, the field 331A displays the color (current sap color) of the concentrated liquid 120 when the concentration ends at the current time 10:41, and the predicted color (predicted sap color) of the maple syrup liquid 125 as a result of distillation to achieve the predetermined sugar content 67% from the current sugar content 35%, respectively provided on the upper and lower sides. The field 331A may display a text “dark” indicating the predicted grade in association with the predicted sap color.

A field 331B displays a combination of the predicted color of the concentrated liquid 120 at the end of the concentration and the predicted sap color when the concentration ends before the predicted end time. Thus, the predicted color of the concentrated liquid 120 at the end of the concentration is the predicted color in a case where the target sugar content of the concentrated liquid 120 is achieved, and the predicted sap color is the predicted color of the maple syrup liquid 125 as a result of distillation to achieve the predetermined sugar content 67% after the concentration.

In the example illustrated in FIG. 7C, the field 331B displays the color of the concentrated liquid 120 when the concentration ends at the predicted end time 16:38, and the predicted color of the maple syrup liquid 125 (predicted sap color) as a result of distillation to achieve the predetermined sugar content 67% from the predicted sugar content (that is, the target sugar content) of the concentrated liquid 120 of 60% at the predicted end time, respectively provided on the upper and lower sides. The field 331B may display a text “golden” indicating the predicted grade in association with the predicted sap color.

A field 331C displays a combination of the predicted color of the concentrated liquid 120 at the end of the concentration and the predicted sap color when the concentration ends at the target end time. Thus, the predicted color of the concentrated liquid 120 at the end of the concentration is the predicted color of the concentrated liquid 120 at the target end time, and the predicted sap color is the predicted color of the maple syrup liquid 125 as a result of distillation to achieve the predetermined sugar content 67% after the concentration.

In the example illustrated in FIG. 7C, the field 331C displays the color of the concentrated liquid 120 when the concentration ends at the target end time 16:00, and the predicted color of the maple syrup liquid 125 (predicted sap color) as a result of distillation to achieve the predetermined sugar content 67% from the predicted sugar content of the concentrated liquid 120 of 52% at the target end time, respectively provided on the upper and lower sides. The field 331C may display a text “amber” indicating the predicted grade in association with the predicted sap color.

Thus, the field 331A described above displays the predicted sap color in a case where the concentration is immediately stopped to be switched to the distillation when the target sugar content is predicted to be unachievable at the predicted end time. The field 331B displays the predicted sap color in a case where the concentration continues to reach the predicted end time (which is after the target end time) until the target sugar content is achieved when the target sugar content is predicted to be unachievable at the predicted end time. The field 331C displays the predicted sap color in a case where the concentration ends at the target end time (even if the target sugar content has not been achieved) to be switched to the distillation when the target sugar content is predicted to be unachievable at the predicted end time.

The color information display section 331 may only display some of the fields 331A to 331C instead of displaying all of them. Alternatively, the color information display section 331 may selectively display any of the fields 331A to 331C.

As described above, with the supporting device 300 of this example, the predicted sap color is displayed for each concentration end timing, whereby options of continuing the concentration or not are presented when the target sugar content is predicted to be unachievable at the predicted end time. Thus, concentration of the maple sap in the production of the maple syrup liquid can be supported.

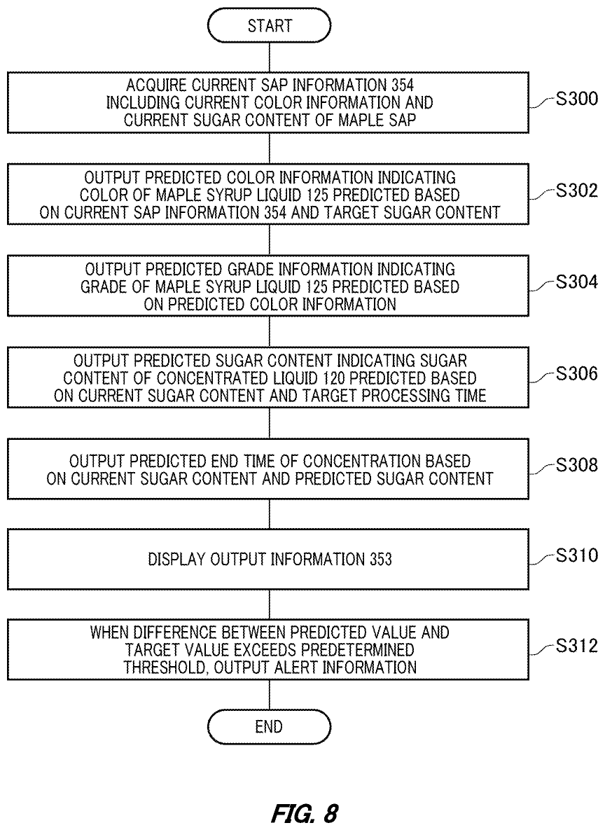

FIG. 8 is a flowchart illustrating an example of a supporting method performed by the supporting device 300. In step S300, the current sap information 354 including the current color information and the current sugar content of the supply liquid 110, which is maple sap, is acquired. In step S302, the predicted color information indicating the color of the maple syrup liquid 125 predicted based on the current sap information 354 and the target sugar content of the concentrated liquid 120 is output.

In step S304, the predicted grade information indicating the grade of the maple syrup liquid 125 predicted based on the predicted color information is output. In step S306, the predicted sugar content indicating the sugar content of the concentrated liquid 120 predicted based on the current sugar content and the target processing time for concentrating the supply liquid 110 into the concentrated liquid 120 is output. In step S308, the predicted end time of the concentration is output based on the current sugar content and the predicted sugar content.

In step S310, the output information 353 may be displayed. When a difference between a predicted value and a target value exceeds a predetermined threshold, alert information may be output in step S312. For example, when the predicted color of the maple syrup liquid 125 is outside the predetermined color range, the color alert information 336 may be displayed.

When the difference between the predicted end time and the target end time exceeds the first time threshold, the time alert information for notifying the user of the deviation of the predicted value from the target value is output, and when the difference between the predicted end time and the target end time exceeds the second time threshold greater than the first time threshold, the change operating condition information is further output as the time alert information. Alternatively, when the difference between the predicted sugar content and the target sugar content exceeds the first sugar content threshold, the sugar content alert information for notifying the user of the deviation of the predicted value from the target value is output, and when the difference between the predicted sugar content and the target sugar content exceeds the second sugar content threshold, the change operating condition information is further output as the sugar content alert information.

In this manner, with the supporting method performed by the supporting device 300 of this example, the status of the concentrated liquid 120 is output based on the current sap information acquired, so that a difference between a predicted status based on the variation of the quality of the supply liquid 110 and the target can be monitored. Furthermore, with the supporting method performed by the supporting device 300 of this example, the operating condition can be changed when the predicted status of the maple syrup liquid 125 deviates from the target. Thus, concentration of the maple sap in the production of the maple syrup liquid can be supported.

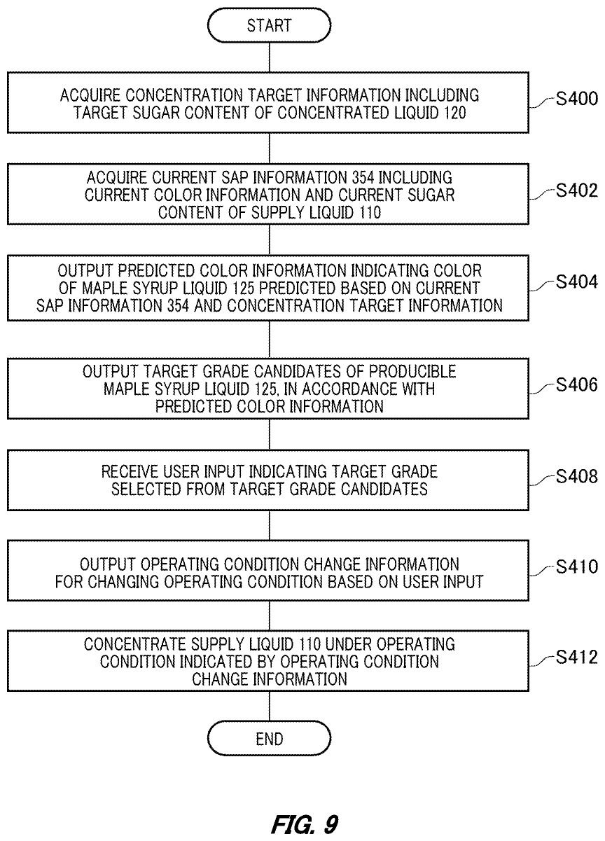

FIG. 9 is a flowchart illustrating an example of a producing method for a maple syrup liquid. In FIG. 6A to FIG. 8, a description is given on the supporting method for supporting the production of the maple syrup liquid using the supporting device 300. In FIG. 9, a producing method for a maple syrup liquid performed using the supporting device 300 while the concentrating device 200 is operating will be described. Here, the concentrating device 200 is concentrating the supply liquid 110 under the selected operating condition.

In step S400, the supporting device 300 acquires the concentration target information including the target sugar content of the concentrated liquid 120. In step S402, the supporting device 300 acquires the current sap information 354 including the current color information and the current sugar content of the supply liquid 110.

In step S404, the supporting device 300 outputs the predicted color information indicating the color of the maple syrup liquid 125 predicted based on the current sap information 354 and the concentration target information.

In step S406, the supporting device 300 outputs the target grade candidates of the producible maple syrup liquid 125, in accordance with the predicted color information. In step S408, the supporting device 300 receives a user input indicating a target grade selected from the target grade candidates. In step S410, the supporting device 300 outputs the operating condition change information for changing the operating condition to the concentrating device 200, based on the user input. In step S412, the concentrating device 200 concentrates the supply liquid 110 under the operating condition indicated by the operating condition change information.

As described above, with the producing method for a maple syrup liquid of this example, the target grade candidates of the producible maple syrup liquid 125 are output in accordance with the predicted color information, so that options for the target grade reflecting the current sap information can be presented to the user. Furthermore, the concentrating device 200 concentrates the supply liquid 110 under the operating condition corresponding to the selected target grade, whereby the maple syrup liquid 125 of a grade reflecting the request of the user can be produced.

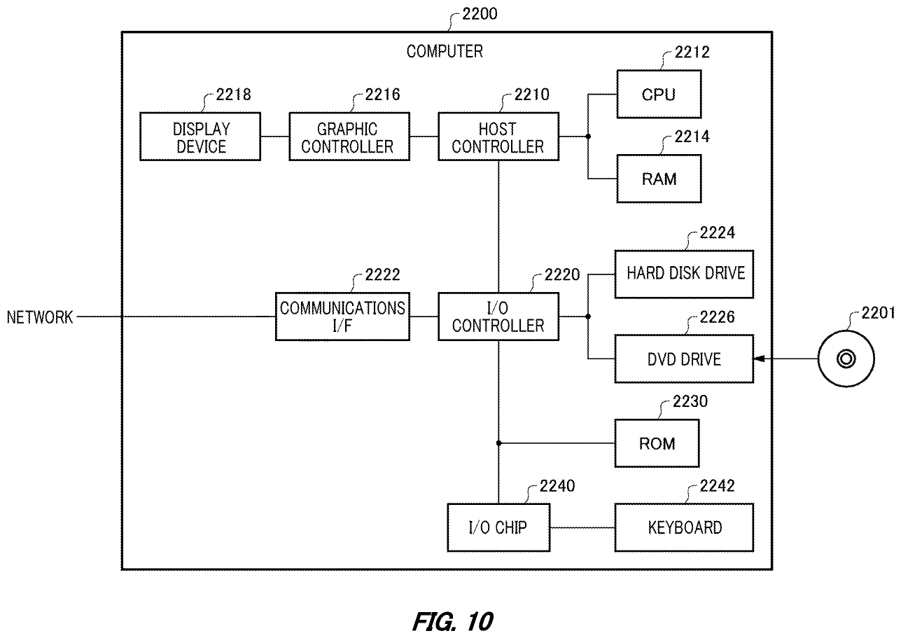

FIG. 10 illustrates an example of a computer 2200 with which some or all of a plurality of aspects of the present invention may be embodied. A program installed in the computer 2200 can make the computer 2200 functions as the operations associated with the device according to an embodiment of the present invention or one or more sections of the device or execute the operation or the one or more sections, and/or make the computer 2200 execute a process according to an embodiment of the present invention or a step of the process. Such a program may be executed by a CPU 2212 to make the computer 2200 execute a predetermined operation associated with some or all of the blocks in the flowchart and the block diagram described in this specification.

The computer 2200 according to the present embodiment includes a CPU 2212, a RAM 2214, a graphic controller 2216, and a display device 2218, which are mutually connected by a host controller 2210. The computer 2200 further includes input/output units such as a communications interface 2222, a hard disk drive 2224, a DVD-ROM drive 2226, and an IC card drive, which are connected to the host controller 2210 via an input/output controller 2220. The computer further includes a legacy input/output unit such as a ROM 2230 and a keyboard 2242 which are connected to the input/output controller 2220 via an input/output chip 2240.

The CPU 2212 operates according to programs stored in the ROM 2230 and the RAM 2214, thereby controlling each unit. The graphics controller 2216 acquires image data generated by the CPU 2212 in a frame buffer and the like provided in the RAM 2214 or in itself, and makes the image data displayed on the display device 2218.

The communication interface 2222 communicates with other electronic devices via a network. The hard disk drive 2224 stores programs and data used by the CPU 2212 within the computer 2200. The DVD-ROM drive 2226 reads the programs or the data from the DVD-ROM 2201, and provides the hard disk drive 2224 with the programs or the data via the RAM 2214. The IC card drive reads the program and data from an IC card, and/or writes the program and data to the IC card.

The ROM 2230 stores therein a boot program or the like executed by the computer 2200 at the time of activation, and/or a program depending on the hardware of the computer 2200. The input/output chip 2240 may also connect various input/output units to the input/output controller 2220 via a parallel port, a serial port, a keyboard port, a mouse port, or the like.

A program is provided by a computer readable medium such as the DVD-ROM 2201 or the IC card. The program is read from a computer readable medium, installed in the hard disk drive 2224, the RAM 2214, or the ROM 2230 which is also an example of the computer readable medium, and is execute by the CPU 2212. Information processing written in these programs is read into the computer 2200, resulting in cooperation between a program and the above-described various types of hardware resources. The apparatus or the method may be configured by realizing an operation or processing on information in accordance with the use of the computer 2200.

For example, when communication is performed between the computer 2200 and an external device, the CPU 2212 may execute a communication program loaded onto the RAM 2214 to instruct communication processing to the communication interface 2222, based on the processing described in the communication program. Under the control by the CPU 2212, the communications interface 2222 reads transmission data stored in a transmission buffer processing region provided in a recording medium such as the RAM 2214, the hard disk drive 2224, the DVD-ROM 2201, or the IC card, transmits the read transmission data to the network, or writes received data received from the network in a reception buffer processing region or the like provided on the recording medium.

In addition, the CPU 2212 may cause all or a necessary portion of a file or a database to be read into the RAM 2214, the file or the database having been stored in an external recording medium such as the hard disk drive 2224, the DVD-ROM drive 2226 (DVD-ROM 2201), the IC card, and the like, and perform various types of processing on the data on the RAM 2214.

The CPU 2212 then writes back the processed data to the external recording medium.

Various types of information such as various types of programs, data, tables, and databases may be stored in recording media and subjected to the information process. The CPU 2212 may execute, on the data read from the RAM 2214, various types of processing including various types of operations, information processing, conditional judgement, conditional branch, unconditional branch, information search/replacement, and the like as described throughout the present disclosure and designated by an instruction sequence of programs, and writes back the result to the RAM 2214. Moreover, the CPU 2212 may search for information in the file, the database, or the like in the recording medium. For example, when a plurality of entries each having an attribute value of a first attribute correlated with an attribute value of a second attribute, are stored in the recording medium, the CPU 2212 may search for an entry matching a condition whose attribute value of the first attribute is designated, from among the plurality of entries, and read the attribute value of the second attribute stored in the entry, thereby acquiring the attribute value of the second attribute correlated with the first attribute that satisfies a predetermined condition.

The programs or a software module described above may be stored on the computer 2200 or in a computer-readable medium near the computer 2200. A recording medium such as a hard disk or a RAM provided in a server system connected to a dedicated communication network or the Internet is usable as the computer readable medium, with which the program is provided to the computer 2200 via the network.

While the embodiments of the present invention have been described, the technical scope of the present invention is not limited to the above described embodiments. It is apparent to persons skilled in the art that various alterations and improvements can be added to the above-described embodiments. It is also apparent from the scope of the claims that the embodiments added with such alterations or improvements can be included in the technical scope of the present invention.

The operations, procedures, steps, and stages of each process performed by an apparatus, system, program, and method shown in the claims, embodiments, or diagrams can be performed in any order as long as the order is not indicated by “prior to,” “before,” or the like and as long as the output from a previous process is not used in a later process. Even if the process flow is described using phrases such as “first” or “next” in the claims, embodiments, or diagrams, it does not necessarily mean that the process must be performed in this order.

### EXPLANATION OF REFERENCES

10: input section, 20: calculation section, 21: quality prediction section, 22: grade determination section, 23: sales prediction section, 24: unit membrane processing calculation section, 25: concentrating device information calculation section, 26: device cost calculation section, 27: process count calculation section, 28: investment return period calculation section, 30: display section, 31: predicted color information display section, 32: predicted color change information display section, 33: predicted grade display section, 34: required processing display section, 35: concentrating device information display section, 36: process count display section, 37: investment return period display section, 40: storage section, 52: input information, 53: output information, 100: supporting device, 110: supply liquid, 120: concentrated liquid, 125: maple syrup liquid, 130: induction solution, 200: concentrating device, 210: supply liquid tank, 215: pump, 220: concentrating section, 222: concentrating module, 225: pump, 230: concentrated liquid tank, 240: induction solution tank, 250: induction solution supply pump, 260: heating section, 270: cooling section, 280: condensed water tank, 290: extraction pump, 295: vacuum pump, 300: supporting device, 320: calculation section, 330: display section, 353: output information, 354: current sap information, 2200: computer, 2201: ROM, 2210: host controller, 2212: CPU, 2214: RAM, 2216: graphics controller, 2218: display device, 2220: output controller, 2222: communications interface, 2224: hard disk drive, 2226: ROM drive, 2230: ROM, 2240: output chip, 2242: keyboard

What is claimed is:

1. A supporting method of supporting concentration of maple sap in production of a maple syrup liquid, the supporting method comprising:
acquiring current sap information including current color information and a current sugar content of the maple sap; and
outputting predicted color information indicating a color of the maple syrup liquid predicted based on the current sap information and a target sugar content of a concentrated liquid obtained by concentrating the maple sap.

2. The supporting method according to claim 1 further comprising outputting predicted grade information indicating a grade of the maple syrup liquid predicted based on the predicted color information.

3. The supporting method according to claim 2 further comprising outputting color alert information when the predicted color of the maple syrup liquid is outside of a predetermined color range.

4. The supporting method according to claim 1 further comprising outputting a predicted sugar content indicating a sugar content of the concentrated liquid predicted based on the current sugar content and a target processing time for concentrating the maple sap into the concentrated liquid.

5. The supporting method according to claim 2 further comprising outputting a predicted sugar content indicating a sugar content of the concentrated liquid predicted based on the current sugar content and a target processing time for concentrating the maple sap into the concentrated liquid.

6. The supporting method according to claim 4 further comprising outputting a predicted end time of the concentration based on the current sugar content and the predicted sugar content.

7. The supporting method according to claim 6 further comprising outputting time alert information when a difference between the predicted end time and a target end time corresponding to the target processing time exceeds a predetermined time threshold.

8. The supporting method according to claim 7, wherein
the time threshold includes a first time threshold and a second time threshold greater than the first time threshold, and
when the difference between the predicted end time and the target end time exceeds the second time threshold, the time alert information further includes operating condition change information.

9. The supporting method according to claim 4 further comprising outputting sugar content alert information when a difference between the predicted sugar content and the target sugar content exceeds a predetermined sugar content threshold.

10. The supporting method according to claim 6 further comprising outputting sugar content alert information when a difference between the predicted sugar content and the target sugar content exceeds a predetermined sugar content threshold.

11. The supporting method according to claim 9, wherein
the sugar content threshold includes a first sugar content threshold and a second sugar content threshold greater than the first sugar content threshold, and
when the difference between the predicted sugar content and the target sugar content exceeds the second sugar content threshold, the sugar content alert information further includes operating condition change information.

12. The supporting method according to claim 4, wherein the predicted color information is information indicating a predicted color of the maple syrup liquid obtained when the concentration ends at any of a current time, a predicted end time at which the sugar content of the maple sap reaches the predicted sugar content, or a target end time corresponding to the target processing time.

13. The supporting method according to claim 6, wherein the predicted color information is information indicating a predicted color of the maple syrup liquid obtained when the concentration ends at any of a current time, a predicted end time at which the sugar content of the maple sap reaches the predicted sugar content, or a target end time corresponding to the target processing time.

14. A producing method for a maple syrup liquid obtained by concentrating maple sap, the producing method comprising:
acquiring concentration target information including a target sugar content of a concentrated liquid obtained by concentrating a supply liquid that is the maple sap;
acquiring current sap information including current color information and a current sugar content of the supply liquid;
outputting predicted color information indicating a color of the maple syrup liquid predicted based on the current sap information and the concentration target information; and
concentrating the supply liquid under a selected operating condition.

15. The producing method according to claim 14 further comprising outputting target grade candidates of the maple syrup liquid that is producible, in accordance with the predicted color information.

16. The producing method according to claim 15 further comprising:
receiving a user input indicating a target grade selected from the target grade candidates;
outputting operating condition change information for changing the operating condition based on the user input; and
concentrating the supply liquid under an operating condition indicated by the operating condition change information.

17. A non-transitory computer readable storage medium having stored thereon a program which causes a computer to perform a supporting method of supporting concentration of maple sap in production of a maple syrup liquid, the supporting method comprising:
acquiring current sap information including current color information and a current sugar content of the maple sap; and
outputting predicted color information indicating a color of the maple syrup liquid predicted based on the current sap information and a target sugar content of a concentrated liquid obtained by concentrating the maple sap.

18. A non-transitory computer readable storage medium having stored thereon a program which causes a computer to perform a producing method for a maple syrup liquid obtained by concentrating maple sap, the producing method comprising:
acquiring concentration target information including a target sugar content of a concentrated liquid obtained by concentrating a supply liquid that is the maple sap;
acquiring current sap information including current color information and a current sugar content of the supply liquid;
outputting predicted color information indicating a color of the maple syrup liquid predicted based on the current sap information and the concentration target information; and
concentrating the supply liquid under a selected operating condition.

19. A supporting device comprising a calculation section that performs the supporting method according to claim 1.

20. A supporting device comprising a calculation section that performs the producing method according to claim 14.
